(19) United States
(12) Patent Application Publication
(10) Pub. No.: US 2005/0203532 A1
(43) Pub. Date: Sep. 15, 2005

(54) Technique and instrumentation for intervertebral prosthesis implantation using independent landmarks

(75) Inventors: Joe W. Ferguson, Collierville, TN (US); Carlos E. Gil, Collierville, TN (US); Toney Ray Owsley, Southaven, MS (US); Leonard Tokish JR., Issaquah, WA (US)

Correspondence Address: HAYNES AND BOONE, LLP, 901 MAIN STREET, SUITE 3100, DALLAS, TX, 75202, US

(73) Assignee: SDGI Holdings, Inc., Wilmington, DE (US)

(21) Appl. No.: 10/799,178

(22) Filed: Mar. 12, 2004

(51) Int. Cl. A61B 17/90; A61F 2/46

(52) U.S. Cl. 606/90; 606/102

(57) ABSTRACT

An assembly for preparing an intervertebral disc space between a first vertebra and a second vertebra to receive a prosthesis comprises a distractor having a first distraction arm and a second distraction arm. The assembly further includes a first anchoring device attached to both the first distraction arm and the first vertebra and a second anchoring device attached to both the second distraction arm and the second vertebra. In this assembly, the first anchoring device moves independently of the second anchoring device.

### BACKGROUND

Recently, technical advances in the design of joint reconstructive devices has revolutionized the treatment of degenerative joint disease, moving the standard of care from arthrodesis to arthroplasty. Reconstruction of a damaged joint with a functional joint prosthesis to provide motion and to reduce deterioration of the adjacent bone and adjacent joints is a desirable treatment option for many patients. For the surgeon performing the joint reconstruction, specialized instrumentation and surgical methods may be useful to facilitate precise placement of the prosthesis.

### SUMMARY

In one embodiment, an assembly for preparing an intervertebral disc space between a first vertebra and a second vertebra to receive a prosthesis comprises a distractor having a first distraction arm and a second distraction arm. The assembly further includes a first anchoring device attached to both the first distraction arm and the first vertebra and a second anchoring device attached to both the second distraction arm and the second vertebra. In this assembly, the first anchoring device moves independently of the second anchoring device.

In another embodiment, a method of preparing an intervertebral disc space, between first and second vertebral bodies of a vertebral column, to receive an intervertebral prosthesis comprises fixedly attaching first and second anchoring devices to the first and second vertebral bodies, respectively. The method further comprises attaching a distraction assembly to the first and second anchoring devices, wherein a first arm of the distraction assembly is attached to the first anchoring device and a second arm of the distraction assembly is attached to the second anchoring device. The method also comprises moving the first and second arms of the distraction assembly, in parallel, relative to one another. The method further comprises independently moving the first and second anchoring devices relative to the first and second arms, respectively.

### BRIEF DESCRIPTION OF THE DRAWINGS

FIG. 1 is a sagittal view of a vertebral column having a damaged disc.

FIG. 2 is a flowchart describing a surgical technique.

FIG. 3 is an isometric view of an alignment guide according to an embodiment of the current disclosure.

FIG. 4 is an isometric view of a distractor assembly according to a one embodiment of the current disclosure.

FIG. 5 is an anchoring device according to an embodiment of the current disclosure.

FIG. 6 is an anchoring device according to still another embodiment of the current disclosure.

FIG. 7 is the distractor assembly of FIG. 4 configured with the anchoring devices of FIGS. 5 an 6.

FIG. 8 is the distractor assembly of FIG. 4 configured with the anchoring devices of FIGS. 5 an 6.

FIG. 9 is the distractor assembly of FIG. 4 configured with the anchoring devices of FIGS. 5 an 6.

FIG. 10 is the distractor assembly of FIG. 4 configured with the anchoring devices of FIGS. 5 and 6 and the alignment guide of FIG. 3.

FIG. 11 is a front view of a measurement instrument according to one embodiment of the current disclosure.

FIG. 12 is an environmental view of the distractor assembly of FIG. 7 and the measurement instrument of FIG. 11.

FIG. 13 is an exploded view of a cutting assembly according to one embodiment of the current disclosure.

FIG. 14 is an environmental view of the cutting assembly of FIG. 13 in operation.

FIG. 15 is an isometric view of a distractor assembly according to a second embodiment of the current disclosure.

FIG. 16 is an anchoring device according to still another embodiment of the current disclosure.

FIG. 17 is an environmental view of the distractor assembly of FIG. 15 coupled with the anchoring device of FIG. 16.

FIG. 18 is an isometric view of a distractor assembly according to still another embodiment of the current disclosure coupled to an anchoring device according to still another embodiment of the current disclosure.

FIG. 19 is an isometric view of a pair of anchoring devices according to still another embodiment of the current disclosure.

### DETAILED DESCRIPTION

The present disclosure relates generally to the field of orthopedic surgery, and more particularly to instrumentation and methods for vertebral reconstruction using an intervertebral prosthesis. For the purposes of promoting an understanding of the principles of the invention, reference will now be made to embodiments or examples illustrated in the drawings, and specific language will be used to describe the same. It will nevertheless be understood that no limitation of the scope of the invention is thereby intended. Any alteration and further modifications in the described embodiments, and any further applications of the principles of the invention as described herein are contemplated as would normally occur to one skilled in the art to which the invention relates.

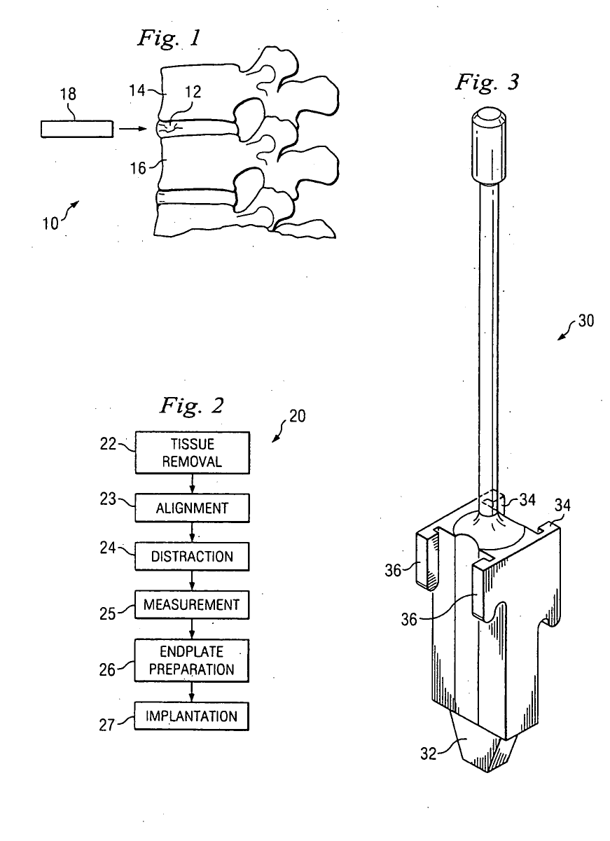

Referring first to FIG. 1, the numeral 10 refers to a human anatomy having a joint location which in this example includes an injured, diseased, or otherwise damaged intervertebral disc 12 extending between vertebrae 14, 16. The damaged disc may be replaced by an intervertebral disc prosthesis 18 which may be a variety of devices including the prostheses which have been described in U.S. Pat. Nos. 5,674,296; 5,865,846; 6,156,067; 6,001,130 and in U.S. Patent Application Nos. 2002/0035400; 2002/0128715; and 2003/0135277 which are incorporated by reference herein.

A surgical technique for repairing the damaged joint may be represented, in one embodiment, by the flowchart 20 depicted in FIG. 2. Referring first to step 22, all or a portion of the damaged disc 12 may be excised. This procedure may be performed using an anterior, anterolateral, lateral, or other approach known to one skilled in the art, however, the following embodiments will be directed toward a generally anterior approach. Generally, the tissue removal procedure 22 may include positioning and stabilizing the patient. Fluoroscopic or other imaging methods may be used to assist with vertebral alignment and surgical guidance. Imaging techniques may also be used to determine the proper sizing of the intervertebral prosthesis 18. In one embodiment, a sizing template may be used to pre-operatively determine the correct prosthesis size. The tissue surrounding the disc space may be retracted to access and verify the target disc space. The area of the target disc may be prepared by removing excess bone, including osteophytes which may have developed, and other tissues which may include portions of the annulus and all or portions of the nucleus pulpous. The tissue removal procedure 22, which may include a discectomy procedure, may alternatively or additionally be performed after alignment and/or measurement procedures have been taken.

Proceeding to step 23 of FIG. 2, various alignment procedures may be conducted to align the intervertebral space in preparation for the disc prosthesis 18. The transverse center of the disc space may be determined and marked. Referring now to FIG. 3, an alignment guide 30, comprising an intervertebral portion 32, may be selected. The intervertebral portion 32 may be selected to permit insertion between the adjacent vertebrae 14, 16 with minimal distraction. The alignment guide may further comprise positioning guides 34, 36. In one embodiment, as illustrated in FIG. 3, the positioning guides 34, 36 may have differing lengths to facilitate easy coupling to subsequent instrumentation.

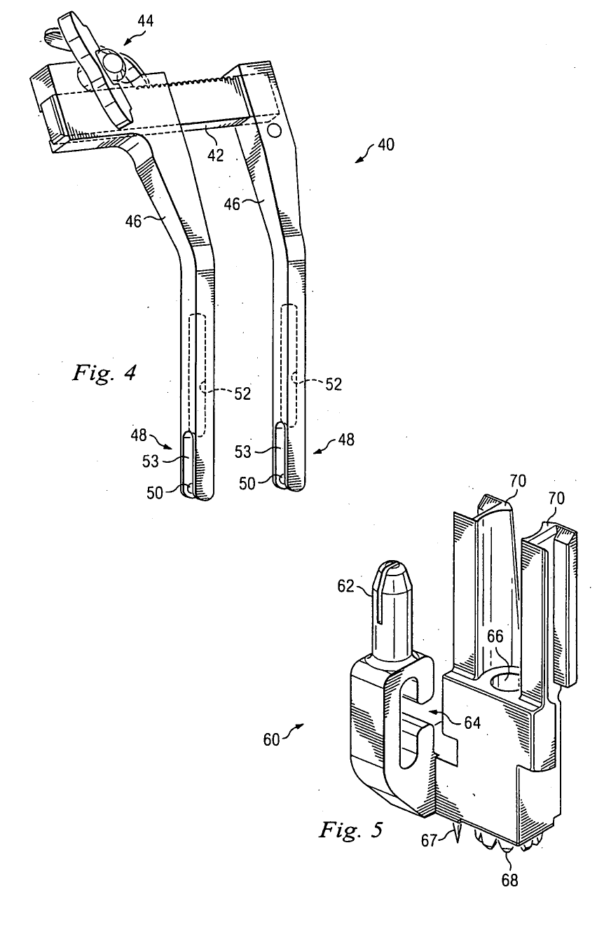

Referring now to FIG. 4, the alignment step 23 (FIG. 2) continues with the introduction of a distractor assembly 40. The distractor assembly 40 may include a cross bar member 42 having a securing mechanism 44. A pair of distracting arms 46 may be attached to the cross bar member 42. A variety of securing mechanisms 44 may be used to maintain a selected distance between the distracting arms 46 including a ratchet system, clamps, threaded connectors, pins, gripping hardware, or other fasteners may be selected as the means to maintain a selected distance between the distracting arms 46. At least one of the distracting arms 46 may be movably connected to cross bar member 42 with the securing mechanism 44. Each of the distracting arms 46 may include attachment mechanisms 48. In the embodiment of FIG. 4, the attachment mechanisms 48 includes pins 50 and hollow recesses 52. In some embodiments, as shown, the one or more of the walls of the hollow recesses 52 may have elongated openings 53. The attachment mechanisms 48 may be used to locate, hold, and/or guide anchoring devices as will be described below. The attachment mechanisms 48 may include stops or other features useful for position verification or instrument support.

Referring now to FIG. 5, an anchoring device 60 may include a connecting portion 62, a pivot mechanism 64, a vertebral body attachment portion 66, a restraint pin 67, a seat 68, and constraint members 70. The anchoring device 60 may attach to one of the distracting arms 46 by engaging the pin 50 with the pivot mechanism 64 and by inserting the connecting portion 62 into one of the hollow recesses 52.

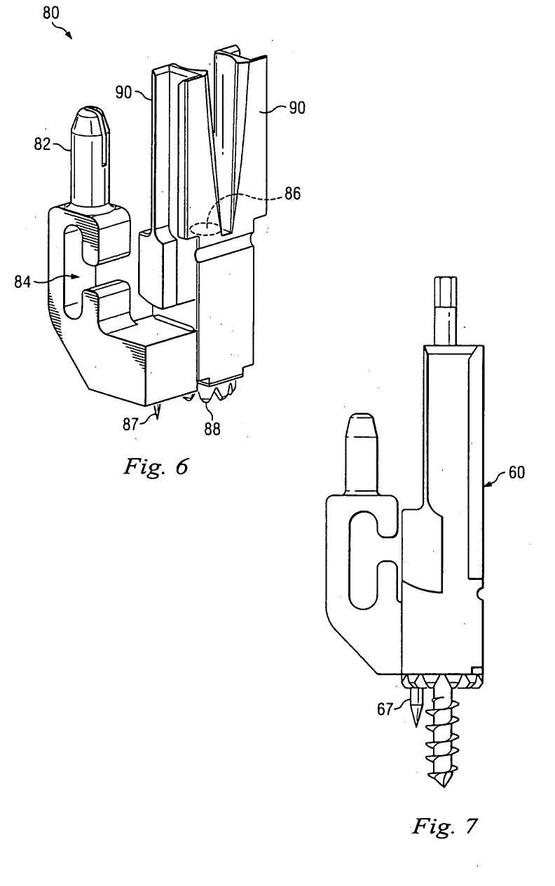

Referring now to FIG. 6, an anchoring device 80, which may complementary to the anchoring device 60 may include a connecting portion 82, a pivot mechanism 84, a vertebral body attachment portion 86, a restrait pin 87, a seat 88, and constraint members 90. The anchoring device 80 may attach to one of the distracting arms 46 by engaging the pin 50 with the pivot mechanism 84 and by inserting the connecting portion 82 into one of the hollow recesses 52. In some embodiments, the anchoring devices may be identical rather than complementary.

Referring now to FIG. 7, the rotation restraint pin 67 of anchoring device 60 is more clearly illustrated. In this embodiment the restraint pins 67, 87 may be retractable, but in other embodiments, the restraint pins may be fixed.

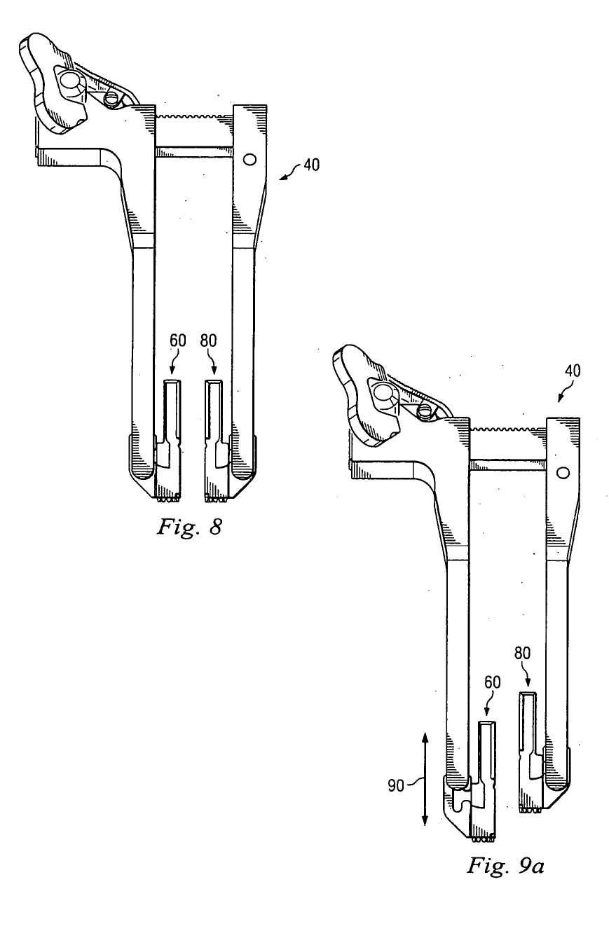

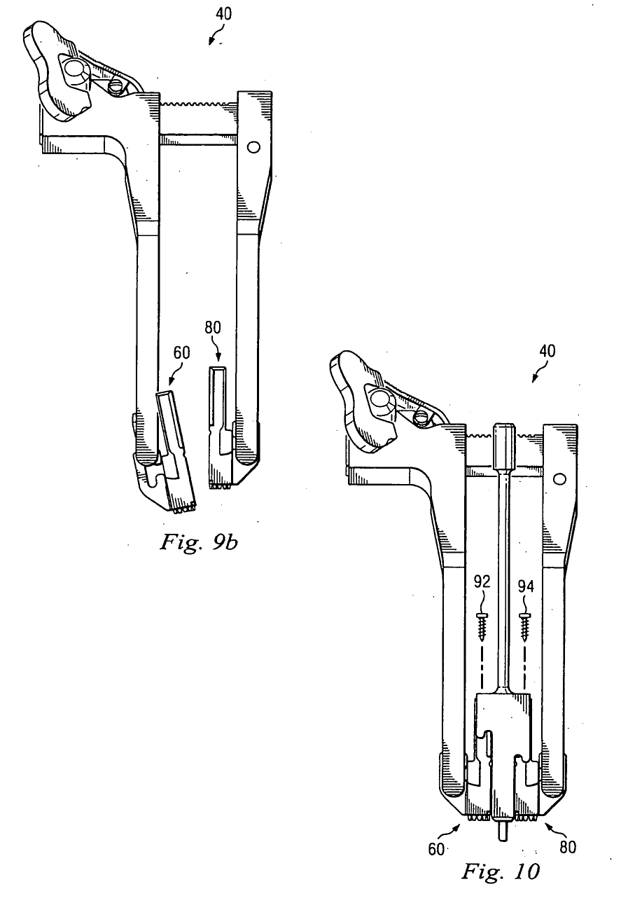

Referring now to FIGS. 8, 9a, and 9b, in this embodiment, the pivot mechanisms 64, 84 are “C”-shaped which may permit independent displacement of the anchoring devices 60, 80 relative to one another along an axis 90 aligned with the axis of the hollow recess 52. When using an anterior surgical technique, the axis 90 may be an anterior-posterior axis. Referring to FIG. 9b, the “C”-shape of the pivot mechanisms 64, 84 may also permit the anchoring devices 60, 80 to independently pivot or rotate in a sagittal plane about the pins 50. In this embodiment, the connecting portions 62, 82 may be pulled from the hollow recesses 52. As the anchoring devices 60, 80 pivot independently of each other, the connecting portions 62, 82 may be permitted to pivot in and out of the elongated openings 53 of the distracting arms 46.

Referring now to FIG. 10, the alignment guide 30 may be coupled to the anchoring devices 60, 80. Specifically, in the illustrated embodiment, one set of positioning guides, for example guides 34, may mate with the constraint portions 90. Then, the second set of positioning guides 36 may mate with the constraint portions 70. The differing lengths of the positioning guides 34, 36 may allow the surgeon to more easily align the positioning guides with the constraint portions. The constraint portions 70, 90 may prevent movement of the alignment guide 30 relative to the anchoring devices 60, 80, respectively.

With the alignment guide 30 coupled to the anchoring devices 60, 80, the intervertebral portion 32 may be inserted between the vertebral endplates of vertebral bodies 14, 16. Alternatively, the insertion of intervertebral portion 32 between the vertebral endplates may take place before or as the alignment guide 30 is coupled to the anchoring devices 60, 80. The anchoring devices 60, 80 may be positioned equidistant from the mid-line center of the intervertebral disc space. Mid-line alignment of the alignment guide 30 may be confirmed, and the sagittal placement of the alignment guide 30 may be assessed with flouroscopic or other imaging techniques. After alignment has been assessed, the alignment guide 30 may be locked in place to either or both of the distractor assembly 40 and the anchoring devices 60, 80. During these alignment procedures, the alignment guide 30 may be generally parallel to the plane of the intervertebral disc space. Additional flouroscopic or other images may be taken throughout the alignment step 23 to verify alignment of the instruments and/or the vertebral bodies.

With the alignment verified, a hole may be drilled into the caudal vertebral body 16 through the vertebral body attachment portion 66 of the anchoring device 60. An anchoring fixture 92, such as a bone screw, may be inserted through the vertebral body attachment portion 66 and into the vertebral body 16 thus firmly locking the seat 68 to the vertebral body 16. As the anchoring fixture 92 descends through the vertebral body attachment portion 66, the anchoring fixture 92 may push on the retractable restraint pin 67, embedding the pin 67 in the vertebral body 16 to prevent rotation of the anchoring device 60 and the subsequent loosening of the anchoring fixture 60 from the vertebral body 16.

The seats 68, 88 of the anchoring devices 60, 80, respectively, may be adjustable and thus may be raised, lowered, and/or tilted. With the seat 68 locked to the vertebral body 16, the seat 88 of the cephalad anchoring device 80 may be adjusted to contact the vertebral body 14, maintaining the alignment guide 30 aligned in a generally anterior-posterior direction. The seat 88 may be adjusted to level the anchoring devices 60, 80, using for example a bubble level (not shown). With the seat 88 in postion, a second hole may be drilled into the cephalad vertebral body 14 through the vertebral body attachment portion 86 of the anchoring device 80. Another anchoring fixture 94, such as a bone screw, may be inserted through the vertebral body attachment portion 86 and into the vertebral body 14 thus firmly locking the seat 88 to the vertebral body 14. As the anchoring fixture 94 descends through the vertebral body attachment portion 86, the anchoring fixture 94 may push on the retractable restraint pin 87, embedding the pin 87 in the vertebral body 14 to prevent rotation of the anchoring device 80 the subsequent loosening of the anchoring fixture 80 from the vertebral body 14. It is understood that in an alternative embodiment, the cephalad anchoring fixture 94 may be placed before the caudal anchoring fixture 92. With the anchoring fixtures 92, 94 in place, the alignment guide 30 may be removed.

Referring again to the surgical technique 20 of FIG. 2, at step 24, distraction may be performed using the distractor assembly 40 (of FIG. 4). With the distractor arms 52, attached to the vertebral bodies 14, 16 by the anchoring devices 80, 60 respectively, the arms 52 may be moved apart placing the vertebral bodies 14, 16 in tension and providing access to the intervertebral space to allow further discectomy and/or decompression procedures as needed. During the distraction, the distractor arms 52 may remain relatively parallel. The securing mechanism 44 may be applied to maintain the vertebral bodies 14, 16 in the desired distracted position.

As the distraction is performed, the connecting portions 62, 82 may remain inside the hollow recesses 52 thereby causing the adjacent endplates of vertebral bodies 14, 16 to remain relatively parallel. Alternatively, during distraction the connecting portions 62, 82 may be pulled from the hollow recesses 52, and the anchoring devices 60, 80 may pivot about pins 50 (as described above) allowing independent movement of the vertebral bodies 14, 16. In some embodiments, the rotation of the vertebral bodies 14, 16 may be constrained to a transversely centered sagittal plane. In other embodiments, the vertebral bodies 14, 16 may rotate in parallel sagittal planes. The independent movement may permit independent preparation of the endplates of vertebral bodies 14, 16 as will be described in detail below. Examples of alternative embodiments which permit full or partial independent movement will also be described below.

With the vertebral bodies 14, 16 distracted and the anchoring devices 60, 80 attached to the vertebral bodies 14, 16, the surgical technique 20 may then proceed to step 25. At step 25, measurements, such as a depth measurement, may be performed at the disc site to determine the proper sizing of instrumentation and devices to be used throughout the remainder of the surgical technique 20.

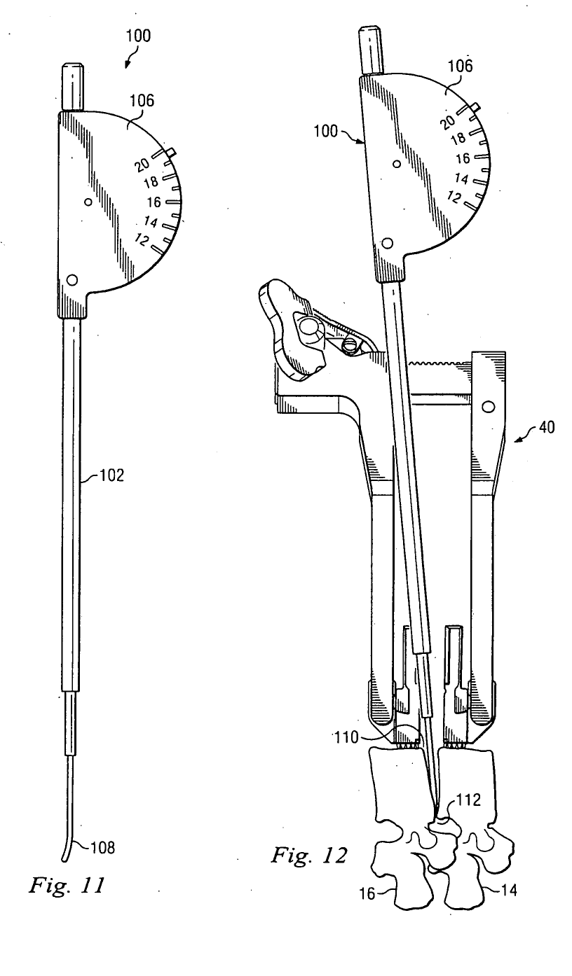

Referring now to FIGS. 11 and 12, the measurement step 25 (FIG. 2) may involve the use of a variety of instrumentation including, for example, a measurement instrument 100 which may assist in the selection of appropriately sized tools to perform subsequent operations such as endplate preparation. In this embodiment, the measurement instrument 100, which includes a shaft 102 extending between an indicator portion 106 and a probe portion 108, may movably or fixedly fasten to one of the anchoring devices 60, 80. The probe portion 108 may travel through the intervertebral disc space to provide a depth measurement. In this embodiment, the indicator portion 106 may indicate the distance from a point, such as an anterior edge 110 of the intervertebral disc space to the posterior margin 102 of the disc space. The indicator portion 106 may magnify the distance traveled by the probe portion 108 providing a measurement which can be used to determine the proper sizing of subsequently used instruments.

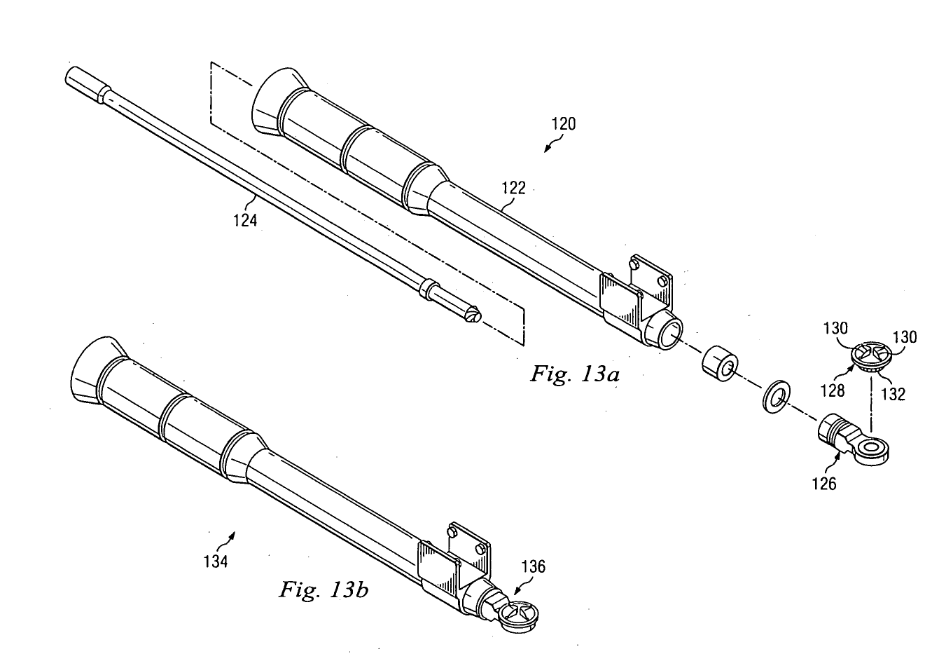

Referring again to FIG. 2, the surgical technique 20 proceeds to step 26 for further preparation of the vertebral endplate surfaces. Referring now to FIG. 13a-13b, to prepare the endplate surfaces to provide a secure seat for the intervertebral prosthesis 18, a cutting instrument may be provided. In the embodiment of FIG. 13a, the cutting instrument 120 may comprise several component parts including an exterior shaft portion 122, an internal shaft portion 124, a cutting head 126, and a cutting device 128. The internal shaft portion 124 may extend through the exterior shaft portion 122 to engage the cutting head 126. The cutting device 128 may be attached to the cutting head 126. The cutting device 128 may have an abrasive surface 130 which can include blades, teeth, a roughened coating or any other surface capable of cutting, abrading, or milling the vertebral endplates. The cutting instrument 120 may include a variety of other components (not shown) such as rivets, bearings, gears, and springs which may be used to assemble the components 122-128 to each other and provide movement to the cutting device 128.

The components 122-128 of the cutting instrument 100 may be constructed to simplify cleaning, promote sterility, enhance reliability, and shorten assembly and surgical time. In one embodiment, the cutting head 1206 may be a single piece of molded polymer. In the embodiment of FIG. 13a, the use of bearings and other components capable of corrosion or susceptible to wearing out easily may be reduced or eliminated. The cutting head 126 may be disposable which can simplify the cleaning of the cutting instrument 120 and may promote sterility in the surgical field. The internal shaft portion 122, which may include an integrated pinion gear, may be disposable to minimize wear on other sensitive components such as gear trains, increasing the reliability of the instrument 120. The use of a pinion shaft as the internal shaft portion 122 may also eliminate bearings and other drive train components which improves the reliability and simplifies cleaning of the cutting instrument 120. The cutting device 128 may be a one-piece metal injection molded cutter having the cutting surface 130 formed on one side and gear teeth 132 integrated into the opposite side to minimize the profile. This integrated embodiment of the cutting device 128 may also promote reliability and sterility.

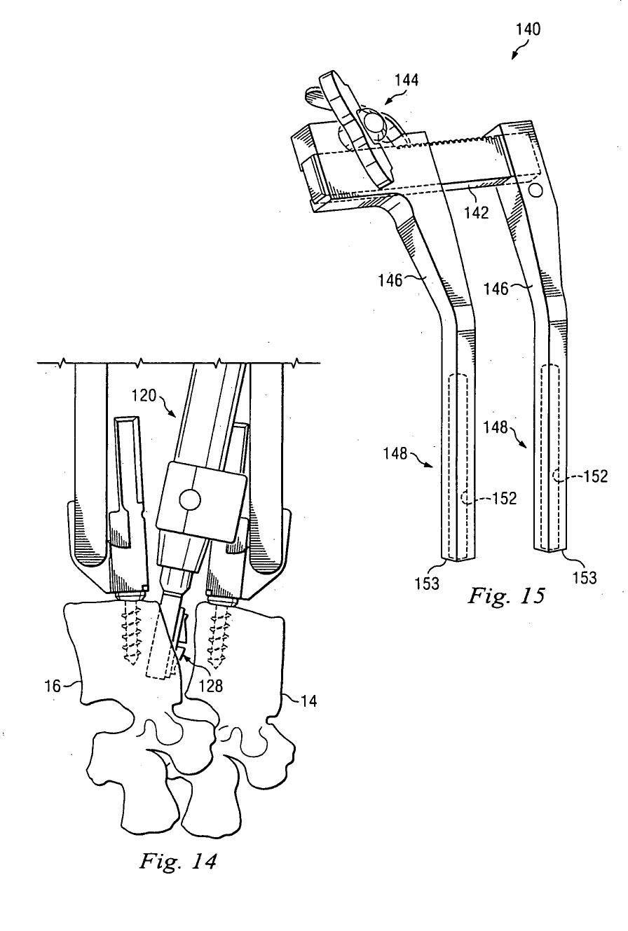

Referring now to FIG. 14, based upon the measurements taken in step 25 and the size and profile of the prosthesis 18 to be implanted, the cutting device 128 may be selected. The cutting instrument 120 may be assembled, as described above, using the selected cutting device 128. With the anchoring devices 60, 80 attached to the distracting arms 46, the cutting instrument 120 may be mounted to one of the anchoring devices 60, 80 such that the cutting device 128 is positioned adjacent to one the vertebral endplates 14, 16. The proper positioning of the cutting device 128 may be established with known offsets and may be verified with fluoroscopic or other imaging techniques. In operation, a power source (not shown) may be provided to the cutting instrument 120 to drive the internal shaft portion 124. The internal shaft portion may directly or indirectly drive the cutting head 126 thereby actuating the cutting device 128. The actuated cutting device 128 causes the cutting surface 130 to shape the selected vertebral endplate. The cutting device 128 or cutting surface 130 may be shaped such that the profile that it creates in the vertebral endplate matches the profile of the selected intervertebral prosthesis 18 to create a secure seat for the prosthesis. After the first endplate is prepared, the cutting instrument 120 may be mounted to the other of the anchoring devices 60, 80 with the cutting device 128 positioned adjacent to the other of the vertebral endplates 14, 16. The cutting instrument 130 may again be powered, this time to shape the second endplate. In this embodiment, as described above, the anchoring devices 60, 80 may remain fixedly aligned to the vertebral bodies and rotatably connected to distracting arms 46. As such, the vertebral bodies 14, 16 may be permitted to rotate independently of each other and therefore, the endplate preparation procedure 26 permits each of the vertebral bodies to be shaped independently.

The cutting instrument described above for FIG. 13a is merely one embodiment which may be used with the distractor assembly 40 and the anchoring devices 60, 80. In alternative embodiments, the cutting device 128 maybe include a burr or other cutting surfaces known in the art. The cutting instrument may also include a telescoping shaft to permit lengthening of the cutting instrument. In some embodiments such as FIG. 13b, the cutting instrument 134 may be comprised largely of reusable components capable of being sterilized, such as by an autoclave. In this embodiment, a cutting head 136 may have a higher profile to accommodate a press-fit gear and other gear train components.

Referring again to FIG. 2, after the vertebral endplates are prepared, the cutting instrument 120 or 134 may be removed from the anchoring device 60 or 80 in preparation for implanting the intervertebral prosthesis 18 at step 27. With the cutting instrumentation removed, the intervertebral prosthesis 18 may be inserted into the prepared space using any of a variety of insertion methods. In some embodiments, the anchoring devices 60, 80 may be used to guide prosthesis insertion instrumentation. After the prosthesis 18 is implanted, the tension on the distractor assembly 40 may be released. The anchoring fixtures 92, 94 may be removed form the vertebral bodies 16, 14 respectively, permitting the distractor assembly 40 to be removed. With all instrumentation removed from the disc site, the wound may be closed.

Referring now to FIG. 15, in an alternative embodiment, a distractor assembly 140 may be used to distract vertebral bodies 14, 16. The distractor assembly 140 may include a cross bar member 142 having a securing mechanism 144. A pair of distracting arms 146 may be attached to the cross bar member 142. A variety of securing mechanisms 144 may be used to maintain a selected distance between the distracting arms 146 including a ratchet system, clamps, threaded connectors, pins, gripping hardware, or other fasteners may be selected as the means to maintain a selected distance between the distracting arms 146. At least one of the distracting arms 146 may be movably connected to cross bar member 142 with the securing mechanism 144. Each of the distracting arms 146 may include attachment mechanisms 148. In the embodiment of FIG. 15, the attachment mechanisms 148 include hollow cavities 152. In some embodiments, as shown, the distracting arms 146 may have relatively flat end portions 153, but in alternative embodiments, the end portions may be angled or curved. The attachment mechanisms 148 may be used to locate, hold, and/or guide anchoring devices as will be described below. The attachment guides 148 may include stops or other features useful for position verification or instrument support.

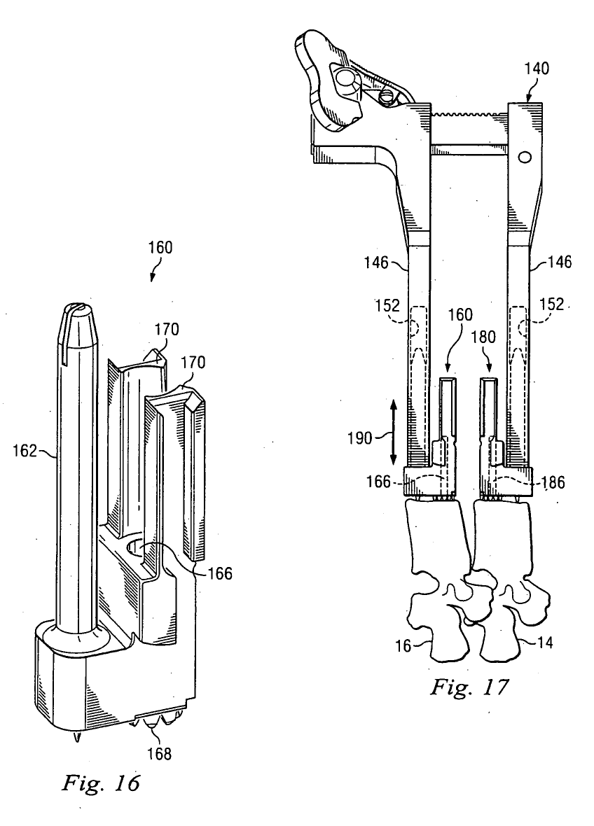

Referring now to FIG. 16-17, an anchoring device 160 may include a connecting portion 162, a vertebral body attachment portion 166, a seat 168, and constraint portions 170. The anchoring device 160 may attach to one of the distracting arms 46 by inserting the connecting portion 162 into one of the hollow cavities 152. An opposing anchoring device 180 having the same or similar features anchoring device 160 including an attachment portion 186 may be attached to the other of the distracting arms 146.

The anchoring devices 160, 180 may be of a configuration which attaches to the vertebral bodies 14, 16 and permits independent movement of the vertebral bodies 14, 16 in the sagittal plane while maintaining alignment of the vertebral bodies 14, 16 in the transverse and coronal planes. The independent movement may permit independent preparation of the endplates of vertebral bodies 14, 16 as will be described in detail below. Examples of alternative embodiments which permit full or partial independent movement will also be described below.

With the anchoring devices 160, 180 connected to the distractor assembly 140 as described above, movement of the vertebral bodies 14, 16 in the sagittal plane may be permitted. As movement occurs, the anchoring devices 160, 180 may maintain a fixed alignment with the vertebral bodies 16, 14. In this embodiment, movement of the attachment portions 166,186 within the hollow cavities 152 may permit independent displacement of the anchoring devices 60, 80 relative to one another along an axis 190 in the sagittal plane. When using an anterior surgical technique, the axis 190 may be an anterior-posterior axis. Using this alternative distractor assembly 140 and anchoring devices 160, 180, the operations of alignment, distraction, measurement, endplate preparation, and implantation may proceed in a fashion similar to that described above in surgical technique 20. In this embodiment, however, the vertebral bodies 14, 16 may be constrained from pivotal movement in the sagittal plane, resulting in a parallel distraction of the vertebral bodies.

A variety of alternative anchoring devices with alternative means for attaching to a distractor assembly may be selected which permit at least some movement of the vertebral bodies 14, 16 in a single plane, such as a sagittal plane. In some embodiments, the connection between the distractor assembly and the anchoring devices may be selectably fixed, pivotable, or movable in a linear direction.

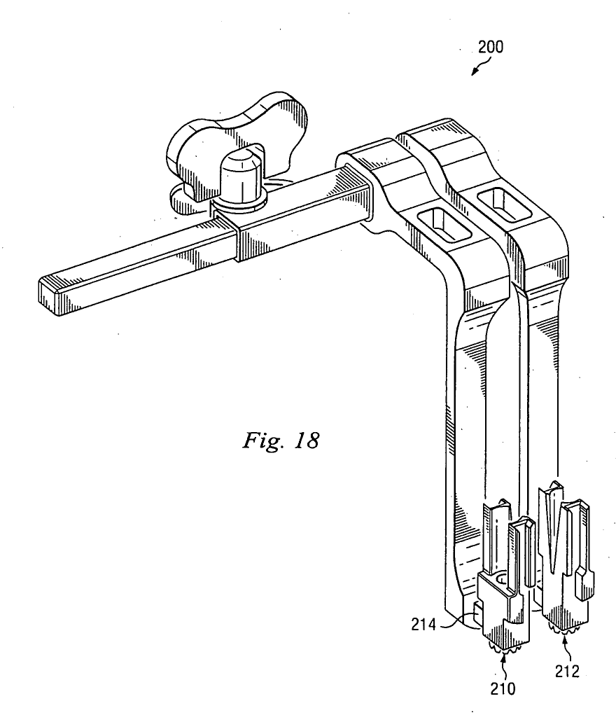

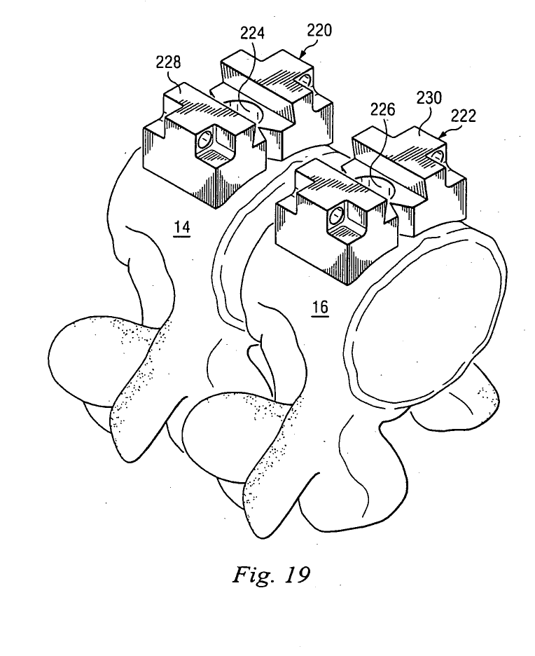

Referring now to FIG. 18, in still another embodiment, a distractor assembly 200 and anchoring devices 210, 212 may be movably connected by a connector 214. The connector 214 may permit rotational movement or linear movement in a single plane, such as a sagittal plane.

Referring now to FIG. 19, in still another embodiment, a pair of anchoring devices 220, 222 may be connected to vertebral bodies 14, 16, respectively. The anchoring devices 220, 222 may include vertebral body attachment apertures 224, 226 and may further include connection portions 228, 230. The connection portions 228, 230 may be used for attaching and/or aligning instrumentation used for measuring, bone preparation, or prosthesis insertion. The anchoring devices 220, 222 may permit independent movement of the vertebral bodies 14, 16 during preparation of the intervertebral site.

Although only a few exemplary embodiments of this invention have been described in detail above, those skilled in the art will readily appreciate that many modifications are possible in the exemplary embodiments without materially departing from the novel teachings and advantages of this invention. Accordingly, all such modifications are intended to be included within the scope of this invention as defined in the following claims. In the claims, means-plus-function clauses are intended to cover the structures described herein as performing the recited function and not only structural equivalents, but also equivalent structures.

1. An assembly for preparing an intervertebral disc space between a first vertebra and a second vertebra to receive a prosthesis, the assembly comprising:
a distractor comprising a first distraction arm and a second distraction arm;
a first anchoring device attached to both the first distraction arm and the first vertebra; and
a second anchoring device attached to both the second distraction arm and the second vertebra,
wherein the first anchoring device moves independently of the second anchoring device.

2. The assembly of claim 1 wherein the movement of the first anchoring device is in a sagittal plane

3. The assembly of claim 2, wherein the movement of the first anchoring device is a pivotal movement.

4. The assembly of claim 1, wherein the movement of the first anchoring device is a linear movement in an anterior-posterior direction.

5. The assembly of claim 1 wherein the first anchoring device comprises a pivot mechanism and the first distracting arm comprises a pin, and further wherein the pivot mechanism engages the pin permitting at least limited rotation of the first anchoring device.

6. The assembly of claim 1 wherein the first anchoring device comprises an elongated shaft and the first distracting arm comprises an elongated recess and further wherein the elongated shaft slidably engages the elongated recess.

7. The assembly of claim 1 further comprising:
an alignment guide interposed between the first and second anchoring devices for sagitally aligning the first and second anchoring devices.

8. The assembly of claim 7 wherein the alignment guide comprises a pair of apertures through which a pair of fasteners may pass to fasten to the vertebral bodies.

9. The assembly of claim 7 wherein the first anchoring device comprises a restraint pin deployable into the first vertebral body as one of the fasteners is passed through one of the apertures and fastened to the first vertebral body.

10. The assembly of claim 1 wherein the first anchoring device comprises an adjustable seat for leveling the anchoring devices.

11. The assembly of claim 1 further comprising:
a measurement instrument attached to the first anchoring device.

12. The assembly of claim 1 further comprising:
a shaping instrument attached to the first anchoring device.

13. A method of preparing an intervertebral disc space, between first and second vertebral bodies of a vertebral column, to receive an intervertebral prosthesis, the method comprising:
fixedly attaching first and second anchoring devices to the first and second vertebral bodies, respectively;
attaching a distraction assembly to the first and second anchoring devices, wherein a first arm of the distraction assembly is attached to the first anchoring device and a second arm of the distraction assembly is attached to the second anchoring device;
moving the first and second arms of the distraction assembly, in parallel, relative to one another;
independently moving the first and second anchoring devices relative to the first and second arms, respectively.

14. The method of claim 13 further comprising shaping a first endplate of the first vertebral body independently of shaping a second endplate of a second vertebral body.

15. The method of claim 14 further comprising attaching a shaping instrument to the first distractor arm prior to shaping the first endplate.

16. The method of claim 13 wherein the first anchoring device independently pivots about a rotation pin in the first distractor arm.

17. The method of claim 13 wherein the first anchoring device independently pivots about a connector extending from the first distractor arm.

18. The method of claim 13 wherein the positioning of the first and second anchoring devices is in a sagittal plane.

19. The method of claim 18 wherein the independent movement of the first and second anchoring devices is in the sagittal plane.

20. The method of claim 13 wherein the first and second anchoring devices are fixedly attached to the first and second bodies equidistant from the center of the intervertebral disc space.

21. An assembly for preparing an intervertebral disc space between first and second vertebral bodies to receive a prosthesis, the assembly comprising:
a distractor, wherein the distractor comprises a first distracting arm in parallel relation to a second distracting arm;
a first anchoring device coupled between the first distracting arm and the first vertebral body, wherein the first anchoring device comprises a first pivot mechanism and the first distracting arm comprises a first pivot pin and further wherein the first pivot mechanism pivotally engages first pivot pin; and
a second anchoring device coupled between the second distracting arm and the second vertebral body, wherein the second anchoring device comprises a second pivot mechanism and the second distracting arm comprises a second pivot pin and further wherein the second pivot mechanism pivotally engages second pivot pin.

22. The assembly of claim 21 further comprising an alignment guide extending between the first and second anchoring devices.

23. The assembly of claim 22 further comprising a milling instrument pivotally attached to the instrumentation guide.
